(19) United States
(12) Patent Application Publication
(10) Pub. No.: US 2023/0410309 A1
(43) Pub. Date: Dec. 21, 2023

(54) METHODS, COMPUTER PROGRAMS, AND SYSTEMS FOR AUTOMATED MICROINJECTION

(71) Applicant: Overture Life, Inc., New York, NY (US)

(72) Inventors: Adrián ALVAREZ FERNANDEZ, Madrid (ES); Santiago MUNNE, Barcelona (ES); Luis MOLLINEDO HERRERA, Almería (ES); Gloria CALDERON OYA, Barcelona (ES); Nuno Luis COSTA BORGES, Barcelona (ES); Jesús RAMOS MEMBRIVE, Madrid (ES); Sergi MAS SABATES, Teià (ES)

(73) Assignee: Overture Life, Inc., New York, NY (US)

(21) Appl. No.: 18/198,501

(22) Filed: May 17, 2023

Related U.S. Application Data
(60) Provisional application No. 63/342,793, filed on May 17, 2022.

(51) Int. Cl. G06T 7/00; G06T 7/30; G06V 20/69; G06V 20/70; G06V 10/82; G06T 7/70; G06T 7/20; G06V 10/774; G16H 30/40; C12N 15/89

(52) U.S. Cl. CPC G06T 7/0014; G06T 2207/30024; G06V 20/698; G06V 20/70; G06V 10/82; G06T 7/70; G06T 7/20; G06V 10/774; G16H 30/40; C12N 15/89; G06T 2207/30044; G06T 2207/20084; G06T 2207/10056; G06T 2207/20036; G06V 2201/034; G06T 2207/20081; G06T 7/30

(57) ABSTRACT

Provided herein are methods, computer programs, and systems for automated microinjection, for example, automated Intracytoplasmic Sperm Injection (ICSI). Methods described herein include creating, by a processing unit, a first dataset of an oocyte and a holding device and a second dataset of an injection pipette; detecting the oocyte and the holding device in the first dataset and the injection pipette in the second dataset; selecting the image of the first dataset and of the second dataset where an equatorial plane of the oocyte/holding device and of the injection pipette has an improved focusing parameter; selecting images of the first and second datasets and labeling the pixels associated with the oocyte and to the injection pipette; detecting a tip of the injection pipette; detecting different morphological structures of the oocyte using artificial intelligence computer vision algorithms on the first dataset; creating an injection trajectory for the injection pipette to perform the ICSI using the detected morphological structures; detecting when the oocyte is rupturing and when the spermatozoa has been released from the injection pipette into the cytoplasm of oocyte.

### CROSS-REFERENCE TO RELATED APPLICATIONS

This application claims the benefit of U.S. Provisional Application No. 63/342,793, filed May 17, 2022, which is incorporated herein by reference in its entirety.

### BACKGROUND

Microinjection is a process used to introduce exogenous substances into cells using a fine-tipped needle, such as a micropipette. Compared to other delivery methods such as electroporation and viral vectors, microinjection can have higher success rates and can better preserve the biological activity of cells. Conventional manual microinjection techniques can be time-consuming and error-prone. As such, automated microinjection systems and methods can enhance process efficiency and injection success rates.

### SUMMARY

Provided herein are methods, computer programs, and systems for automated microinjection, for example, automated Intracytoplasmic Sperm Injection (ICSI). The methods, computer programs, and systems can include a series of computer vision (CV) detection algorithms and training thereof to execute a microinjection procedure. ICSI is a procedure in which a single spermatozoon is directly injected into an oocyte cytoplasm using micromanipulators, micropipettes, and biochips.

Methods described herein include performing an ICSI procedure using CV. In some embodiments, methods described herein do not include using CV in combination with electrical resistance or pressure sensing.

In some aspects, provided herein is an automated method performing an ICSI procedure, comprising one or more of the following steps:
a) receiving, by a processing unit, a first set of images of an oocyte and of holding device (e.g., a holding pipette or a biochip) used to immobilize or hold the oocyte, creating a first dataset as a result, the oocyte being attached to the holding device, the first set of images being acquired by means of moving the oocyte using a motor in an axis perpendicular to an optical sensor plane, and each of the images of the first set of images is associated with a given oocyte motor position;
b) receiving, by the processing unit, a second set of images of an injection pipette (e.g., a microinjection pipette), creating a second dataset as a result, the second set of images being acquired by moving the injection pipette in an axis perpendicular to the optical sensor plane, and each of the images of the second set of images being associated with a given pipette motor position;
c) detecting, by the processing unit, the oocyte and/or the holding pipette in the first dataset and the injection pipette in the second dataset using one or more CV detection algorithms;
d) selecting, by the processing unit using artificial intelligence techniques, the image of the first dataset in which the oocyte is most in focus in comparison to the other images of the first set of images, wherein the image coincides with the equatorial plane of the oocyte;
e) positioning, by the motor, the oocyte to the motor position associated with the image of the first dataset in which the oocyte is most in focus in comparison to the other images of the first set of images;
f) selecting, by the processing unit, a plurality of images of the first dataset and labeling the pixels that belong to the holding device and to the oocyte, and selecting a plurality of images of the second dataset and labeling the pixels that belong to the injection pipette;
g) detecting, by the processing unit, a tip of the injection pipette by implementing one or more CV algorithms on the second dataset using the labeled pixels of the pipette tip;
h) detecting, by the processing unit, different morphological structures of the oocyte by implementing one or more CV algorithms on the first dataset, the different morphological structures such as a zona pellucida, a polar body, a perivitelline space, and a cytoplasm; and
i) creating, by the processing unit, a trajectory for the injection pipette in order to perform ICSI using the detected morphological structures of the oocyte;
j) executing the trajectory of i) which includes movement of the injection pipette (to the motor position);
k) perforating, by a laser or a piezo, the oocyte outer layer (zona pellucida) to expel the chunk of the zona pellucida to the outside of the oocyte;
l) detecting, by the processing unit, expulsion of a chunk of zona pellucida (cork) using one or more CV algorithms;
m) executing of piercing of the oolemma (oocyte membrane) using an injection pipette associated with a piezo and detection of rupture of the oolemma using one or more CV algorithms;
n) depositing, by the injection pipette, a spermatozoon inside the oocyte; and
o) detecting, by the processing unit, expulsion of the spermatozoon using one or more CV algorithms.

After step e), selecting, by the processing unit, the image of the second dataset where the injection pipette tip is most in focus, using artificial intelligence techniques, and using the position of the motor associated with the most focused image, the injection pipette is moved to its given position which results in alignment of the oocyte equatorial plane with the tip of the injection pipette.

In some embodiments, an image or set of images is acquired by an imaging device, such as an optical instrument, an optical sensor, a microscope, a camera, or any device capable of forming an image and that comprises an optical system capable of forming a digital image.

In some embodiments, in step a) the first set of images and the second set of images are acquired separately.

In some embodiments, in step a) the first set of images and the second set of images are acquired from a lower side of the oocyte to an upper side of the oocyte.

In some embodiments, step a) further comprises randomly selecting a plurality of images of the first and second datasets and labeling the oocyte and/or the holding pipette and the injection pipette in the randomly selected images using an image detection algorithm, e.g., a region of interest (ROI) algorithm.

In some embodiments, in step f) the at least one artificial neural network (ANN) and/or the at least one CV algorithm are implemented on the image of the first dataset having a maximum value of a focusing parameter, for instance the variance of the Laplacian, among others.

In some embodiments, step f) further comprises detecting a background of the oocyte.

In some embodiments, the trajectory is created by computing a center of the cell morphology structure in a given image, calculating where the trajectory crosses the zona pellucida and how much it must penetrate the cytoplasm using the computed center, and checking if the trajectory crosses the polar body.

In some embodiments, ICSI can be executed by actuation of high frequency vibrations of the injection pipette (using a piezo actuator) that achieves a drilling effect on the zona pellucida and punctures the oolemma when the injection pipette follows the calculated trajectory.

The piercing of the outer shell of the oocyte (e.g., zona pellucida perforation) can be performed by using a laser to disintegrate the oocyte membrane with heat, or applying by an injection pipette high frequency vibrations on the oocyte membrane (PIEZO method), i.e., piezo-assisted ICSI (Piezo-ICSI).

In some embodiments, the piezo is deactivated as the piezo crosses the perivitelline space. The piezo can then be reactivated again when the injection pipette has sufficiently pushed the oolemma into the cytoplasm and successfully punctures the oolemma.

In some embodiments, the method further comprises acquiring a third set of images of the executed ICSI, creating a third dataset as a result, labeling images into two classes:
a) a first class where the oolemma is rupturing or relaxing; and
b) a second class where the oolemma is not rupturing or relaxing.

The classification can be performed by a CV algorithm from consecutive images and using these as input for training a classification algorithm.

In some embodiments, the method further comprises detecting when a spermatozoon is expelled from the injection pipette by means of acquiring a fourth set of images of the executed ICSI using an optical sensor, creating a fourth dataset as a result, labeling the spermatozoon using an image detection algorithm, labeling the injection pipette using an image detection algorithm, predicting where the spermatozoon is by training a detection CV algorithm using the fourth dataset, and predicting where the injection pipette is by training a detection CV algorithm using the fourth dataset. The image detection algorithm can be a ROI or semantic segmentation algorithm. Each image of the fourth set of images independently contains the spermatozoon during performance of the ICSI maneuver including when the pipette is removed from the oocyte.

Further provided herein is a system for automation of an ICSI maneuver, comprising an optical sensor, a holding device adapted to contain an oocyte, an injection pipette, and a processing unit comprising at least one memory and one or more processors. The one or more processors are configured to:
a) receive a first set of images of the oocyte and of the holding pipette, creating a first dataset as a result, the first set of images being acquired by means of the oocyte/holding pipette moving in a perpendicular axis to an optical sensor plane, and each of the images of the first set of images being associated with a given oocyte/holding pipette motor position; and a second set of images of the injection pipette, creating a second dataset as a result, the second set of images being acquired by moving the pipette in the perpendicular axis to the sensor plane, and each of the images of the second set of images being associated with a given pipette motor position;
b) detect the oocyte and/or the holding pipette in the first dataset and the injection pipette in the second dataset using one or more CV detection algorithms;
c) select the image of the first dataset and of the second dataset where an equatorial plane of the oocyte and/or the holding pipette/device and of the injection pipette tip are most in focus, and using the position of the oocyte and holding pipette/device motors associated with the selected images to align the oocyte and the injection pipette using artificial intelligence techniques;
d) select a plurality of images of the first dataset and labeling the pixels that belong to the holding pipette and to the oocyte, and selecting a plurality of images of the second dataset and labeling the pixels that belong to the injection pipette;
e) detecting a tip of the injection pipette by implementing a semantic segmentation algorithm on the labelled pixels;
f) detect different morphological structures of the oocyte by implementing at least one Artificial Neural Network and/or at least one CV algorithm on the first dataset, the different morphological structures including: a zona pellucida, a polar body, a perivitelline space, and a cytoplasm; and
g) create a trajectory for the injection pipette in order to perform the ICSI using the detected morphological structures of the oocyte by calculating where the end of the pipette tip ends up for release of the spermatozoa into the oocyte.

Other embodiments disclosed herein also include software programs to perform methods and operations summarized above and disclosed in detail below. For example, provided herein is a computer program product having a computer-readable medium including computer program instructions encoded thereon that when executed on at least one processor in a computer system causes the processor to perform the operations described herein.

### BRIEF DESCRIPTION OF THE DRAWINGS

FIG. 1 is an illustration of a stack of images of the oocyte and the holding pipette. The horizontal black lines illustrate the different focal planes.

FIG. 2 is an illustration of an example of the ROI labels of the oocyte and holding pipette.

FIG. 3 is a below (panel A) and side view (panel B) illustration of an ICSI maneuver. Arrows show the direction of movement of the holding pipette/device or injection pipette.

FIG. 4 is a flowchart illustrating an embodiment of the proposed method.

### DETAILED DESCRIPTION

Provided herein are methods, systems, and computer programs for automated microinjection, for example, automated ICSI. In some embodiments, automated ICSI is performed using only CV strategies.

The metaphase II (MII) is the stage during oocyte maturation in which the first polar body is extruded. The MII oocyte includes three main components: zona pellucida (ZP), which is a protective glycoprotein space that encapsulates the oocyte; ooplasm area, which is the cytoplasm of the oocyte; and the perivitelline space (PVS), the thick layer between the ooplasm and the ZP. Morphology of an oocyte can be an essential indicator of the embryo's potential for successful implantation and healthy development after ICSI. Morphological structures can include the zona pellucida, the polar body, the oolemma, the perivitelline space, and the cytoplasm. Morphological characteristics can include oocyte area, oocyte shape, ooplasm area, ooplasm translucency, zona pellucida thickness, and perivitelline space width.

In some embodiments, the CV algorithms can be used to:
Align in an axis perpendicular to an optical sensor plane an injection pipette 300 (or injection pipette) and oocyte 100 using ANN and CV algorithms.
Detect the image formed by the optical instrument, the injection pipette 300 and oocyte 100 using ANN and CV algorithms.
Detect different morphological structures 101-105 of the oocyte 100 (e.g., zona pellucida 105, polar body 104, oolemma 103, perivitelline space 102, and cytoplasm 101 (see FIG. 3).
Create an injection trajectory 301 for the injection pipette 300 such that the injection pipette 300 will not damage the polar body 104 upon injection and also use a piezo actuator to penetrate the zona pellucida 105 and oolemma 103.

Various aspects of the method described herein can be embodied in programming. Program aspects of the technology can be products or articles of manufacture, for example, in the form of executable code and/or associated data that is carried on or embodied in a type of machine readable medium. Tangible non-transitory storage-type media can include a memory or other storage for the computers, processors, or the like, or associated modules thereof, such as semiconductor memories, tape drives, disk drives, and the like, which can provide storage for the software programming.

All or portions of the software can be communicated through a network such as the Internet or various other telecommunication networks. Such communications, for example, can enable loading of the software from one computer or processor into another, for example, from a management server or host computer of a scheduling system into the hardware platform(s) of a computing environment or other system implementing a computing environment or similar functionalities in connection with image processing. Thus, another type of media that can bear the software elements can include optical waves, electrical waves, and electromagnetic waves, such as used across physical interfaces between local devices, through wired and optical landline networks and over various air-links. The physical elements that carry such waves, such as wired or wireless links, optical links or the like, also can be considered as media bearing the software. Computer or machine-readable medium can refer to a medium that participates in providing instructions to a processor for execution.

A machine-readable medium can take many forms, including but not limited to, a tangible storage medium, a carrier wave medium, or physical transmission medium. Non-volatile storage media include, for example, optical or magnetic disks, such as any of the storage devices in any computer(s), or the like, which may be used to implement the system or any components thereof. Volatile storage media can include dynamic memory, such as a main memory of such a computer platform. Tangible transmission media can include coaxial cables, and copper wire and fiber optics, including the wires that form a bus within a computer system. Carrier-wave transmission media can take the form of electric or electromagnetic signals, or acoustic or light waves such as those generated during radio frequency (RF) and infrared (IR) data communications. Common forms of computer-readable media can include, for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a PROM and EPROM, a FLASH-EPROM, a NAND flash, SSD, any other memory chip or cartridge, a carrier wave transporting data or instructions, cables or links transporting such a carrier wave, or any other medium from which a computer can read programming code and/or data. These forms of computer readable media can be involved in carrying one or more sequences of one or more instructions to a physical processor for execution.

Although the implementation of various components described herein may be embodied in a hardware device, it may also be implemented as a software only solution, e.g., an installation on an existing server. In addition, image processing can be implemented as a firmware, hardware, software, e.g., application programs, or a combination thereof.

### EXAMPLES

### Example 1: An Automated ICSI Method and System for Execution Thereof

CV algorithms described herein provide the automatization of an ICSI procedure. In this example, the AI computer vision algorithms are referred to as ALGX_AI. The classical computer vision algorithms are referred to as ALGX_CV.

### Oocyte and Holding Pipette Detector (ALG1_AI)

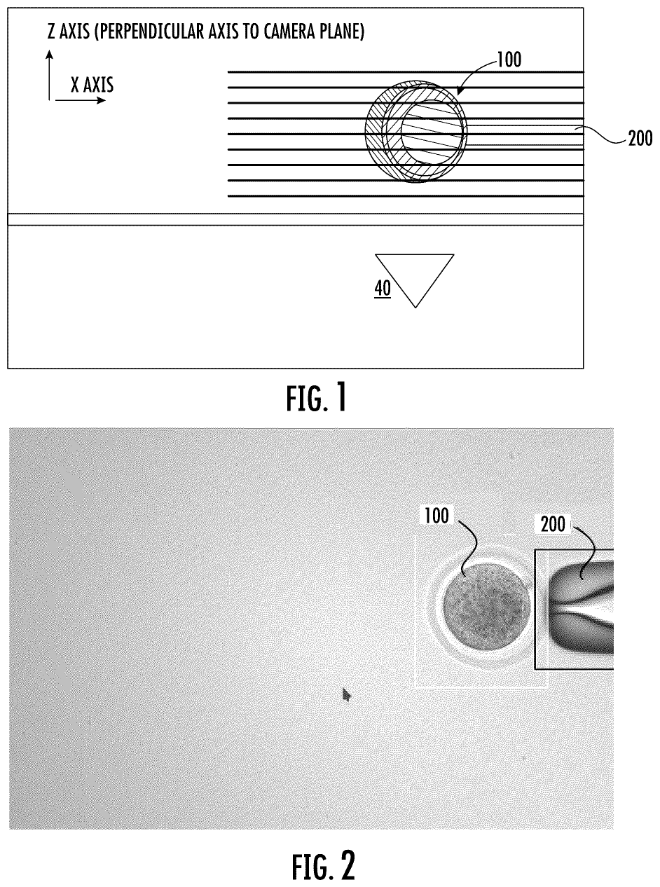

FIG. 1 illustrates an example of how images of an oocyte and a holding pipette are acquired. An oocyte 100 is attached to a holding pipette 200. A stack (or series) of N number of images of the oocyte 100 and the holding pipette 200 (or a first pipette) in the optical field of view 40 are created. For each oocyte 100, a stack of images is collected starting from the underside (lower end) of the oocyte 100 to the upper side (upper end) of the oocyte 100, thereby generating a first dataset, i.e., dataset_oocyte_and_holding_pipette.

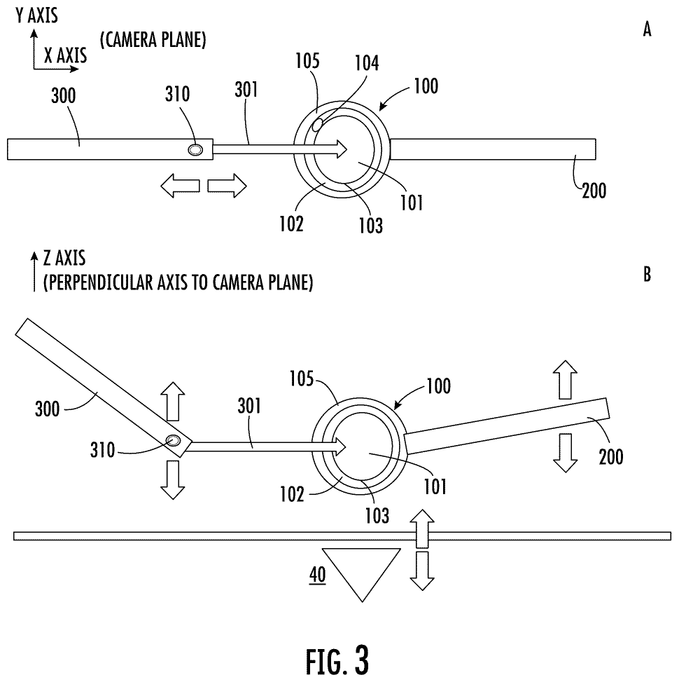

N pictures per stack are then randomly selected and pixels of the oocyte 100 and holding pipette 200 are labeled using ROI. FIG. 2 illustrates an example of how images are acquired.

The labeled data are then used to train a detection algorithm. For example, the data are split into three groups: 80% train, 10% validation, and 10% test. The algorithm is trained until the loss of the validation is stable.

To evaluate the performance of this process, a test set was used. The test set was selected from the dataset randomly. To evaluate the accuracy, the intersection over union of 75% or greater can be used. Results: Accuracy: 100% (train set size=80, validation set size=10, and test set size=10).

### Oocyte Plane Selector (ALG2_CV)

For each stack, the oocyte 100 is cropped with fixed dimensions. To identify the equatorial plane of the oocyte 100, ALG1_AI can be used. In some cases, this step can be performed manually.

For each image of the stack, a gaussian blur with a kernel of K×K pixels is applied, and a focusing parameter (e.g., the variance of the Laplacian) is then calculated.

The image of the stack in which the equatorial plane is best focused is identified by selecting the image of the stack where the focusing parameter is greatest.

The performance of this automated process was evaluated by comparing the output results with results manually determined by an embryologist. A senior embryologist manually identified the equatorial plane in 40 sets of stacks. The output of the embryologist and ALG2_CV were then compared. The experiment is successful when both outputs are the same. Results: Accuracy 100% in the 40 experiments.

### Injection Pipette Detector (ALG3_AI)

FIG. 3 illustrates an example of how images of the injection pipette are acquired. A stack of N Number of images of the injection pipette 300 was created. For each injection pipette 300, a stack of pictures is collected starting from the lower side (lower end) of the oocyte 100 to the upper side (upper end), thereby generating an injection dataset, i.e., dataset_injection_pipette.

N pictures per stack are randomly selected and pixels that belong to the injection pipette 300 are labeled using ROI.

The labeled data are then used to train a semantic segmentation algorithm. For example, the data are split into three groups: 80% train, 10% validation, and 10% test. Data augmentation is then carried out in the train set to increase the data diversity. Finally, the algorithm is trained until the loss of the validation is stable.

To evaluate the performance of this process, a test set is used. To evaluate the accuracy, the f1 score in images of 768×768 pixels can be used. Results: f1_score_pipette=0.06 (train set size=300, validation set size=30, and test set size=30).

### Injection Pipette Plane Selector and Tip Detection (ALG4_CV)

The injection pipette tip can be used to align the injection pipette to the oocyte. Focusing only on the injection pipette tip can be advantageous because the whole pipette is not parallel to the optical sensor. To align the injection pipette to the oocyte, only the tip of the injection pipette 300 needs to be in the same plane as the equatorial plane of the oocyte 100 as illustrated in FIG. 3.

For each stack, a crop of the image is made with size of the M×M at the center of the pipette's tip. To identify the tip of the injection pipette 300, ALG3_AI can be used. In some cases, this step can be performed manually.

For each image of the stacks, a gaussian blur with a kernel of K×K is applied, and a focusing parameter (e.g., the variance of the Laplacian) is then calculated.

The image of the stacks in which the tip of the injection pipette 300 is best focused is identified by selecting the image of the stack where the focusing parameter is maximum.

ALG3_AI is then used to detect the location of the tip of the injection pipette 300.

The performance of this automated process was also evaluated by comparing the output results with results manually determined by an embryologist. A senior embryologist manually labeled the image where the oocyte is most in focus and localized the injection pipette tip in 40 sets of stacks. The output of the embryologist was then compared to the output of ALG4_CV. When both outputs are the same or the error of the tip of the pipette is less than 5 pixels, the experiment is considered successful. Note: 5 pixels in our system (standard microscope) which is equivalent to less than 1 μm of distance. Results: Accuracy 100% in the 40 experiments.

### Morphology Detector (ALG5_AI)

From the first dataset, the image of the oocyte 100 is cropped and the image where the focusing parameter is maximum in each stack is selected.

An expert embryologist labels all the pixels that belong to the polar body 104, perivitelline space 102, cytoplasm 101, and zona pellucida 105. All other pixels are labeled as background.

The labeled data are then used to train a semantic segmentation algorithm. First, data augmentation is performed and then the data are split into three groups: 80% train, 10% validation, and 10% test. The algorithm is trained until the loss of the validation is stable.

To evaluate the performance of this process, a test set was used. To evaluate the accuracy, the f1 score in images of 768×768 pixels is used. Results: f1_score_pb=0.4, f1_score_PVS=0.2, f1_score_Cell=0.01 y f1_score_ZP=0.05 (train set size=80, validation set size=10, and test set size=10).

### Morphology Post-Processing (ALG6_CV)

Using the output of the ALG5_AI, all pixels belonging to the same class are grouped together forming a blob. Once all the blobs of the same class are created the following steps are performed:
a) Of the blobs associated with the perivitelline space 102, the cytoplasm 101, and the zona pellucida 105, only the largest blob for each is kept; the remaining blobs are assigned as background.
b) Of the blobs associated with the polar body 104, only the largest blob is kept, and the area of the blob (in pixels) is calculated. If the area is greater than a threshold, then this blob is labeled as the polar body 104. Otherwise, the blob is labeled as the perivitelline space 102/cytoplasm 101.

An injection trajectory 301 is created using the blobs from the previous step as input.
a. Y axis: The cytoplasm blob is centered on the Y axis.
b. X axis: Using the Y-axis from the previous step, an injection trajectory 301 in which the injection pipette 300 crosses the zona pellucida 105 and the penetration distance into the cytoplasm blob are calculated. Whether the trajectory 301 crosses the polar body 104 or approaches too close to the polar body 104 is determined. This information can be used to abort the injection or continue with the procedure.
c. Piezo actuator: First, a piezo actuator is actuated when the injection pipette 300 crosses the zona pellucida 105, after which the piezo actuator is shut down as the piezo actuator crosses the perivitelline space 102. At this point, the tip of the injection pipette 300 contacts and pushes the oolemma 103. Finally, the piezo actuator is activated again when the injection pipette 300 penetrates deep enough to rupture the oolemma 103.

The polar body detection rate was 90% and the detection of the other morphological features was 100% with error lower than 1% in a test set of 10 pictures. To evaluate the performance of this process, a test set is used. To evaluate the accuracy of the blobs construction an experiment is considered successful when the Intersection over Union (IoU) of the blobs with the labels are higher than 97%.

The accuracy of the trajectory was evaluated by comparing the output results with results manually determined by an embryologist. A senior embryologist manually created the injection trajectory for 40 oocytes. The test is considered successful when the difference of the trajectories is lower than 5 pixels. Results: Blob accuracy: 100% zona pellucida, 100% perivitelline space, 100% cytoplasm, and 90% polar body. Trajectory accuracy: 97.5%. The higher the number of labels, the better the results.

### Oolemma Relaxation Detector (ALG7_AI)

N videos are collected from a full injection, thereby generating a third dataset, i.e., dataset_pipette_penetration.

An embryologist classifies and labels the frames from the dataset in two classes:
a. Class 0: when the oolemma 103 is rupturing and retracting; and
b. Class 1: when condition of class 0 is not true.

The optical flow from consecutive frames is computed and the images are labelled using previous labels as follows:
a. Class 0: when one of the frames of the oolemma 103 is labeled as 0 (oolemma is rupturing and retracting); and
b. Class 1: when condition of class 0 is not true.

In some cases, the oolemma is not sufficiently ruptured during execution of an ICSI procedure. Determining whether the oolemma is ruptured can be necessary to determine when to deactivate the perforation device (e.g., a laser or piezo) and initiate release of the spermatozoon into the oocyte. Optical flow can be determined by one or more AI or CV algorithms, e.g., a Gunnar-Farneback algorithm.

A classification algorithm is then trained using the labeled computed optical flow. Data augmentation can then be performed. Then, the data can be split into three groups: 80% train, 10% validation, and 10% test. The algorithm is trained until the loss of the validation is stable.

To evaluate the performance of this process, a test set is used. When the AI and embryologist outputs are the same or when the AI detects the puncture of the oolemma within 5 frames, the experiment is considered successful. Results: Accuracy=90% (train set size=80, validation set size=10, and test set size=10).

### Sperm and Pipette Tracking (ALG8_AI)

From the third dataset, images of the injection pipette 300, zona pellucida 105, polar body 104, and cytoplasm 101 can be cropped and labeled at the pixel level, as in ALG5_AI. The spermatozoon/sperm 310 can be also labeled using ROI.

Both algorithms are trained as ALG5_AI and as ALG1_AI.

When the spermatozoon 310 surpasses the tip of the injection pipette 300, the system detects that an injection has occurred.

To evaluate the performance of this process, videos that were not used for training are considered. A test is considered successful when the AI and embryologist outputs are the same or when the AI detects the injection of the spermatozoon 310 in the following 2 frames.

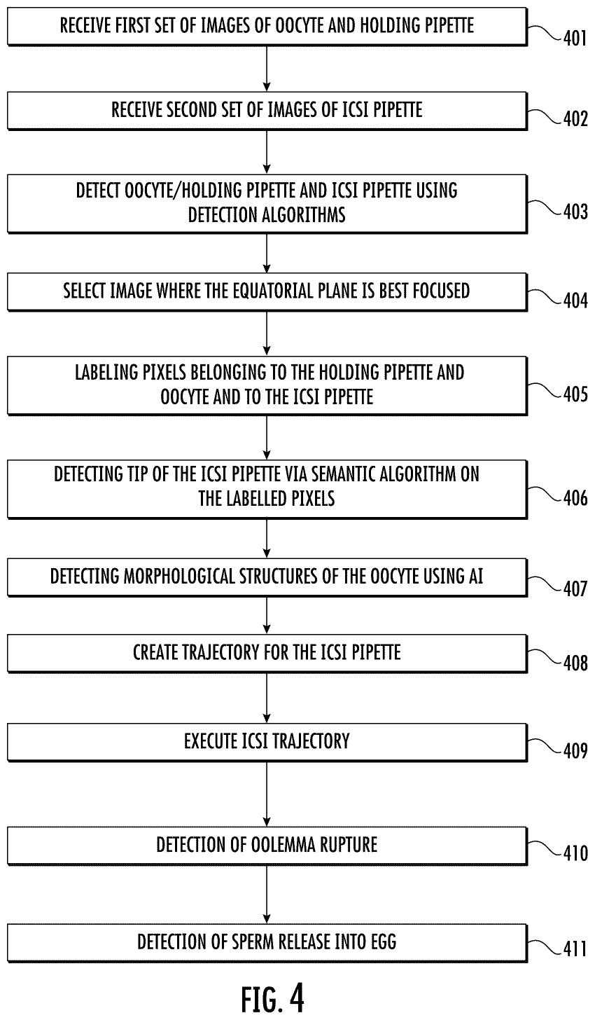

FIG. 4 summarizes the method described herein, which is illustrated in FIG. 3. The process starts when the oocyte 100 is immobilized to the holding pipette 200 and in the field of view of the optical sensor 40, and when the spermatozoon 310 has been loaded in the injection pipette 300 (see FIG. 3). At step 401, a processing unit of the system receives a first set of images (or a first stack of images) of the oocyte 100 and the holding pipette 200. At step 402, the processing unit receives a second set of images (or a second stack of images) of the injection pipette 300. The first and second set of images are acquired by the optical sensor 40 moving in an axis perpendicular to the optical sensor plane, where each of the images of the first set of images and second set of images is associated with a given optical sensor motor position. At step 403, the oocyte 100 and/or the holding pipette 200 in the first dataset are detected and the injection pipette 300 in the second dataset is detected. This detection can be performed by using one or more CV detection algorithms, for instance, the (ALG1_AI) and (ALG2_CV) described above. At step 404, the processing unit selects an image of the first dataset where the equatorial plane of the oocyte 100 and/or the holding pipette 200 has the best focusing parameter, and an image of the second dataset where the equatorial plane of the of the injection pipette 300 has the best focusing parameter. The positions of the sample/oocyte motor associated to the respective selected images can be used to align the oocyte 100 and/or the holding pipette 200 and the injection pipette 300, respectively. At step 405, the pixels associated with the oocyte 100 and/or the holding pipette 200 are labeled and the pixels associated with the injection pipette 300 are labeled. At step 406, the processing unit then detects a tip of the injection pipette 300 by implementing a semantic segmentation algorithm on the labeled pixels. At step 407, the processing unit detects different morphological structures 101-105 of the oocyte 100 by implementing artificial intelligence and/or CV algorithms on the first dataset. At step 408, an injection trajectory 301 for the injection pipette 300 is created to perform the ICSI using the detected morphological structures 101-105 of the oocyte 100. At step 409, the ICSI is executed based on the injection trajectory 301. At step 410, the processing unit detects oolemma rupture in the dataset by classifying images into two groups: ruptured/relaxed oolemma or not ruptured/relaxed oolemma. At step 411, sperm is released into the oocyte.

In some embodiments, a first set of images of the oocyte 100 is collected and the equatorial plane of the oocyte 100 is identified using the ALG1_AI and ALG2_CV algorithms described above; a second set of images of the injection pipette 300 is collected and the most focused image of the second set of images and injection pipette's tip are identified using the ALG3_AI and ALG4_CV algorithms, respectively. Then, the INJECTION trajectory 301 can be created using the ALG6_CV algorithm. The ALG8_AI algorithm can be used to maintain the spermatozoon 310 at the tip of the injection pipette 300 during execution of the injection trajectory 301. When executing the injection trajectory 301, the ALG7_AI and the ALG8_AI algorithms can be used to deliver the spermatozoon 310, e.g., just after the oolemma 103 is punctured.

### EMBODIMENTS

The following non-limiting embodiments provide illustrative examples of the devices, systems, and methods disclosed herein, but do not limit the scope of the disclosure.

Embodiment 1. A method, comprising:
a) receiving a first set of images, wherein each image of the first set of images independently contains an oocyte immobilized by a holding device, wherein the oocyte in each image of the first set of images is the same oocyte, and wherein the holding device in each image of the first set of images is the same holding device;
b) labeling, by an image detection algorithm, a plurality of pixels associated with the oocyte in each image of the first set of images; and
c) determining, using artificial intelligence, and based on the labeled plurality of pixels associated with the oocyte, the image in which the oocyte is most in focus in comparison to the other images of the first set of images.

Embodiment 2. The method of embodiment 1, further comprising:
d) receiving a second set of images, wherein each image of the second set of images independently contains an injection pipette, wherein the injection pipette in each image of the second set of images is the same injection pipette, wherein in each image of the second set of images the injection pipette is positioned for injection of a spermatozoon into the oocyte.
e) labeling, by an image detection algorithm, a plurality of pixels associated with the injection pipette in each image of the second set of images; and
f) determining, using artificial intelligence, and based on the labeled plurality of pixels associated with the injection pipette, the image in which the injection pipette is most in focus in comparison to the other images of the second set of images.

Embodiment 3. The method of embodiment 1 or 2, wherein each image of the first set of images is acquired by an imaging device, wherein each image of the first set of images has a visual plane and the visual plane of each image of the first set of images is parallel, wherein the oocyte moves in an axis perpendicular to an optical sensor plane, wherein each position along the axis perpendicular to the sensor plane is independently associated with a given oocyte position, wherein the sensor plane is parallel to the visual plane of each image of the first set of images, wherein each image of the first set of images is independently associated with an oocyte position, wherein one oocyte position is most effective, wherein the most effective oocyte position is the position associated with the image of the first set of images where the oocyte is most in focus in comparison to the other images of the first set of images.

Embodiment 4. The method of embodiment 2 or 3, wherein each image of the second set of images is acquired by an imaging device along the axis perpendicular to the sensor plane, wherein each position along the axis perpendicular to the sensor plane is independently associated with a given injection pipette position, wherein each image of the second set of images is independently associated with an injection pipette position, wherein one injection pipette position is most effective, wherein the most effective injection pipette position is the position associated with the image of the second set of images where the injection pipette is most in focus in comparison to the other images of the second set of images.

Embodiment 5. The method of any one of embodiments 2-4, further comprising aligning the oocyte and the injection pipette based on: (i) the image of the first set of images where the oocyte is most in focus in comparison to the other images of the first set of images; and (ii) the image of the second set of images where the injection pipette is most in focus in comparison to the other images of the second set of images.

Embodiment 6. The method of any one of embodiments 1-5, further comprising identifying a morphological structure of the oocyte based on the labeled plurality of pixels associated with the oocyte.

Embodiment 7. The method of embodiment 6, wherein the identifying the morphological structure of the oocyte based on the labeled plurality of pixels is by an artificial neural network.

Embodiment 8. The method of embodiment 6 or 7, wherein the identifying the morphological structure of the oocyte based on the labeled plurality of pixels is by a computer vision algorithm.

Embodiment 9. The method of any one of embodiments 1-8, further comprising detecting a background of the oocyte in each image of the first set of images.

Embodiment 10. The method of any one of embodiments 2-9, further comprising identifying a tip of the injection pipette based on the labeled plurality of pixels associated with the injection pipette.

Embodiment 11. The method of any one of embodiments 2-10, wherein each image of the first set of images and each image of the second set of images are acquired from a lower side of the oocyte to an upper side of the oocyte.

Embodiment 12. The method of any one of embodiments 6-11, further comprising determining an injection trajectory into the oocyte for the injection pipette based on the identified morphological structure of the oocyte and the identified tip of the injection pipette.

Embodiment 13. The method of embodiment 12, wherein the injection trajectory is determined by:
identifying a center of the morphological structure of the oocyte;
determining where the injection trajectory crosses a zona pellucida of the oocyte;
determining a distance that the injection trajectory must penetrate into cytoplasm of the oocyte to be effective using the identified center of the morphological structure; and
determining whether the injection trajectory crosses a polar body of the oocyte.

Embodiment 14. The method of embodiment 13, wherein the morphological structure is the zona pellucida.

Embodiment 15. The method of embodiment 13, wherein the morphological structure is the polar body.

Embodiment 16. The method of embodiment 13, wherein the morphological structure is a perivitelline space.

Embodiment 17. The method of embodiment 13, wherein the morphological structure is the cytoplasm.

Embodiment 18. The method of any one of embodiments 12-17, further comprising executing, by the injection pipette, an intracytoplasmic sperm injection (ICSI) on the oocyte at the injection trajectory, wherein the spermatozoon is injected from the injection pipette into the oocyte.

Embodiment 19. The method of embodiment 18, further comprising activating the injection pipette to pierce the zona pellucida when the injection pipette crosses a zona pellucida of the oocyte.

Embodiment 20. The method of embodiment 19, further comprising deactivating the injection pipette when the injection pipette crosses a perivitelline space of the oocyte.

Embodiment 21. The method of embodiment 20, further comprising reactivating the injection pipette to puncture the oolemma, thereby releasing the spermatozoon inside the oocyte.

Embodiment 22. The method of any one of embodiments 2-21, further comprising:
receiving a third set of images, wherein each image of the third set of images independently contains the oocyte during execution of the ICSI, wherein the oocyte comprises an oolemma;
labeling, by an image detection algorithm, a plurality of pixels associated with rupturing or relaxing of the oolemma in each image of the third set of images; and
labeling, by an image detection algorithm, a plurality of pixels associated with the oolemma not rupturing or relaxing of the oolemma in each image of the third set of images.

Embodiment 23. The method of embodiment 22, further comprising calculating the optical flow among consecutive images of the third set of images.

Embodiment 24. The method of embodiment 22 or 23, further comprising training a classification algorithm using the labeled plurality of pixels associated with the oolemma rupturing or relaxing and the labeled plurality of pixels associated with the oolemma not rupturing or relaxing to classify the images in two classes.

Embodiment 25. The method of any one of embodiments 18-24, further comprising detecting release of the spermatozoon from the injection pipette by:
receiving a fourth set of images, wherein each image of the fourth set of images independently contains the spermatozoon during execution of the ICSI on the oocyte at the injection trajectory, wherein the spermatozoon in each image of the fourth set of images is the same spermatozoon, and wherein the injection pipette in each image of the fourth set of images is the same injection pipette;
labeling, by an image detection algorithm, a plurality of pixels associated with the spermatozoon in each image of the fourth set of images;
predicting a location of the spermatozoon by training a detection algorithm using the fourth set of images; and
predicting a location of a tip of the injection pipette by training a detection algorithm using the fourth set of images.

Embodiment 26. A system comprising:
a processing unit comprising at least one memory and one or more processors configured to execute the method of any one of embodiments 1-25.

Embodiment 27. A computer program product comprising a non-transitory computer-readable medium having computer-executable code encoded therein, the computer-executable code adapted to be executed to implement the method of any one of embodiments 1-25.

What is claimed is:

1. A method, comprising:
a) receiving a first set of images, wherein each image of the first set of images independently contains an oocyte immobilized by a holding device, wherein the oocyte in each image of the first set of images is the same oocyte, and wherein the holding device in each image of the first set of images is the same holding device;
b) labeling, by an image detection algorithm, a plurality of pixels associated with the oocyte in each image of the first set of images; and
c) determining, using artificial intelligence, and based on the labeled plurality of pixels associated with the oocyte, the image in which the oocyte is most in focus in comparison to the other images of the first set of images.

2. The method of claim 1, further comprising:
d) receiving a second set of images, wherein each image of the second set of images independently contains an injection pipette, wherein the injection pipette in each image of the second set of images is the same injection pipette, wherein in each image of the second set of images the injection pipette is positioned for injection of a spermatozoon into the oocyte.
e) labeling, by an image detection algorithm, a plurality of pixels associated with the injection pipette in each image of the second set of images; and
f) determining, using artificial intelligence, and based on the labeled plurality of pixels associated with the injection pipette, the image in which the injection pipette is most in focus in comparison to the other images of the second set of images.

3. The method of claim 1, wherein each image of the first set of images is acquired by an imaging device, wherein each image of the first set of images has a visual plane and the visual plane of each image of the first set of images is parallel, wherein the oocyte moves in an axis perpendicular to an optical sensor plane, wherein each position along the axis perpendicular to the sensor plane is independently associated with a given oocyte position, wherein the sensor plane is parallel to the visual plane of each image of the first set of images, wherein each image of the first set of images is independently associated with an oocyte position, wherein one oocyte position is most effective, wherein the most effective oocyte position is the position associated with the image of the first set of images where the oocyte is most in focus in comparison to the other images of the first set of images.

4. The method of claim 2, wherein each image of the second set of images is acquired by an imaging device along the axis perpendicular to the sensor plane, wherein each position along the axis perpendicular to the sensor plane is independently associated with a given injection pipette position, wherein each image of the second set of images is independently associated with an injection pipette position, wherein one injection pipette position is most effective, wherein the most effective injection pipette position is the position associated with the image of the second set of images where the injection pipette is most in focus in comparison to the other images of the second set of images.

5. The method of claim 2, further comprising aligning the oocyte and the injection pipette based on: (i) the image of the first set of images where the oocyte is most in focus in comparison to the other images of the first set of images; and (ii) the image of the second set of images where the injection pipette is most in focus in comparison to the other images of the second set of images.

6. The method of claim 1, further comprising identifying a morphological structure of the oocyte based on the labeled plurality of pixels associated with the oocyte.

7. The method of claim 6, wherein the identifying the morphological structure of the oocyte based on the labeled plurality of pixels is by an artificial neural network.

8. The method of claim 6, wherein the identifying the morphological structure of the oocyte based on the labeled plurality of pixels is by a computer vision algorithm.

9. The method of claim 1, further comprising detecting a background of the oocyte in each image of the first set of images.

10. The method of claim 2, further comprising identifying a tip of the injection pipette based on the labeled plurality of pixels associated with the injection pipette.

11. The method of claim 2, wherein each image of the first set of images and each image of the second set of images are acquired from a lower side of the oocyte to an upper side of the oocyte.

12. The method of claim 6, further comprising determining an injection trajectory into the oocyte for the injection pipette based on the identified morphological structure of the oocyte and the identified tip of the injection pipette.

13. The method of claim 12, wherein the injection trajectory is determined by:
identifying a center of the morphological structure of the oocyte;
determining where the injection trajectory crosses a zona pellucida of the oocyte;
determining a distance that the injection trajectory must penetrate into cytoplasm of the oocyte to be effective using the identified center of the morphological structure; and
determining whether the injection trajectory crosses a polar body of the oocyte.

14. The method of claim 13, wherein the morphological structure is the zona pellucida.

15. The method of claim 13, wherein the morphological structure is the polar body.

16. The method of claim 13, wherein the morphological structure is a perivitelline space.

17. The method of claim 13, wherein the morphological structure is the cytoplasm.

18. The method of claim 12, further comprising executing, by the injection pipette, an intracytoplasmic sperm injection (ICSI) on the oocyte at the injection trajectory, wherein the spermatozoon is injected from the injection pipette into the oocyte.

19. The method of claim 18, further comprising activating the injection pipette to pierce the zona pellucida when the injection pipette crosses a zona pellucida of the oocyte.

20. The method of claim 19, further comprising deactivating the injection pipette when the injection pipette crosses a perivitelline space of the oocyte.

21. The method of claim 20, further comprising reactivating the injection pipette to puncture the oolemma, thereby releasing the spermatozoon inside the oocyte.

22. The method of claim 18, further comprising:
receiving a third set of images, wherein each image of the third set of images independently contains the oocyte during execution of the ICSI, wherein the oocyte comprises an oolemma;
labeling, by an image detection algorithm, a plurality of pixels associated with rupturing or relaxing of the oolemma in each image of the third set of images; and
labeling, by an image detection algorithm, a plurality of pixels associated with the oolemma not rupturing or relaxing of the oolemma in each image of the third set of images.

23. The method of claim 22, further comprising calculating the optical flow among consecutive images of the third set of images.

24. The method of claim 22, further comprising training a classification algorithm using the labeled plurality of pixels associated with the oolemma rupturing or relaxing and the labeled plurality of pixels associated with the oolemma not rupturing or relaxing to classify the images in two classes.

25. The method of claim 18, further comprising detecting release of the spermatozoon from the injection pipette by:
receiving a fourth set of images, wherein each image of the fourth set of images independently contains the spermatozoon during execution of the ICSI on the oocyte at the injection trajectory, wherein the spermatozoon in each image of the fourth set of images is the same spermatozoon, and wherein the injection pipette in each image of the fourth set of images is the same injection pipette;
labeling, by an image detection algorithm, a plurality of pixels associated with the spermatozoon in each image of the fourth set of images;
predicting a location of the spermatozoon by training a detection algorithm using the fourth set of images; and
predicting a location of a tip of the injection pipette by training a detection algorithm using the fourth set of images.

26. A system comprising:
a processing unit comprising at least one memory and one or more processors configured to execute the method of claim 1.

27. A computer program product comprising a non-transitory computer-readable medium having computer-executable code encoded therein, the computer-executable code adapted to be executed to implement the method of claim 1.
